(19) United States
(12) Patent Application Publication
(10) Pub. No.: US 2023/0346431 A1
(43) Pub. Date: Nov. 2, 2023

(54) APPROXIMATION DEVICE

(71) Applicant: Children's Medical Center Corporation, Boston, MA (US)

(72) Inventor: James K. Maclaine, Boston, MA (US)

(73) Assignee: Children's Medical Center Corporation, Boston, MA (US)

(21) Appl. No.: 17/918,807

(22) PCT Filed: Apr. 12, 2021
(86) PCT No.: PCT/US2021/026844

§ 371 Date: Oct. 13, 2022

Related U.S. Application Data
(60) Provisional application No. 63/009,401, filed on Apr. 13, 2020.

(51) Int. Cl. A61B 17/66; A61B 17/64

(52) U.S. Cl. CPC A61B 17/663; A61B 17/6425; A61B 17/6433

(57) ABSTRACT

Methods and devices for approximating jaw and corresponding tissue segments are disclosed herein. In some embodiments, the approximation device includes a first jaw attachable to a first jaw segment, a second jaw attachable to a second jaw segment, the second jaw segment being smaller than the first jaw segment, and a housing. The second jaw is movably attached to the housing, the second jaw configured to move the second jaw segment toward the first jaw segment. The second jaw is configured to translate between a first position and a second position during a first segment of travel of the second jaw, and configured to at least rotate between the second position and a third position during a second segment of travel.

### RELATED APPLICATIONS

The present application claims the benefit under 35 U.S.C. § 119(e) of U.S. Provisional Application Ser. No. 63/009,401, filed Apr. 13, 2020 and entitled “APPROXIMATION DEVICE”, the entire contents of which are incorporated by reference herein.

### FIELD

Disclosed embodiments relate to a device for approximating jaw segments and/or tissue portions, such as before a surgical procedure.

### BACKGROUND

An orofacial cleft, an opening in either or both of the gums and lip, is the most common birth defect in infants. In addition to causing a facial abnormality, this condition is associated with a variety of other health issues, including feeding, hearing, and/or speech problems. Infants with an orofacial cleft birth defect typically undergo several reconstructive surgeries early in life, typically within the first three months of birth, to repair the defect as the infant's facial structures develop. Prior to the first surgery, the infant's upper jaw segments may be approximated.

### SUMMARY

According to one embodiment, an approximation apparatus includes a first jaw attachable to a first jaw segment, a second jaw attachable to a second jaw segment, the second jaw segment being smaller than the first jaw segment, and a housing. The second jaw is movably attached to the housing, the second jaw configured to move the second jaw segment toward the first jaw segment. The second jaw is configured to translate between a first position and a second position during a first segment of travel the second jaw. The second jaw is configured to at least rotate between the second position and a third position during a second segment of travel.

According to another embodiment, an approximation device includes a first jaw attachable to a first jaw segment, a second jaw attachable to a second jaw segment, the second jaw arranged to move the second jaw segment toward the jaw segment, and a housing, wherein the first jaw is fixedly attached to the housing and the second jaw is attached to the housing via a rocker arm, the rocker arm being moveable relative to the housing. The rocker arm is configured to translate the second jaw from a first position to a second position during a first segment of travel, and to at least rotate the second jaw from the second position to a third position during a second segment of travel.

According to another embodiment, a method of approximating first and second and jaw segments via an approximation device having first and second jaw attached to a housing is disclosed. The method includes attaching a first jaw to a first jaw segment, the first jaw being attached to a housing, attaching a second jaw to a second jaw segment, the second jaw being moveably attached to the housing, translating the second jaw relative to the housing to move the second jaw from a first position to a second position, and pivoting the second jaw relative to the housing to move the second jaw from the second position to a third position.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a top view of an approximation device attachable to two jaw segments according to some embodiments;

FIG. 2 is a perspective view of an approximation device according to another embodiment;

FIG. 3 is a front view of the approximation device of FIG. 2 attachable to first and second jaw segments;

FIG. 4 is a perspective view of the approximation device of FIG. 1, with first and second jaws shown removed;

FIG. 5 is a top view of the approximation device of FIG. 1, with a second jaw shown in a first position;

FIG. 6 is the approximation device of FIG. 5, with the second jaw shown in a second position;

FIG. 7 is the approximation device of FIG. 5, with the second jaw shown in a third position;

FIG. 8 shows a portion of the approximation device of FIG. 1;

FIG. 9 shows a portion of the approximation device of FIG. 2;

FIG. 10 is a partially exploded view of the approximation device of FIG. 2;

FIG. 11 is a bottom view of the approximation device of FIG. 2, with a second jaw shown in a first position;

FIG. 12 is the approximation device of FIG. 11, with the second jaw in a second position;

FIG. 13 is the approximation device of FIG. 11, with the second jaw in a third position;

FIG. 14 is a perspective view of an approximation device according to another embodiment;

FIG. 15 is a cross-sectional view of the approximation device of FIG. 14, with a second jaw in a first position;

FIG. 16 is the approximation device of FIG. 15, with the second jaw in a second position;

FIG. 17 is the approximation device of FIG. 15, with the second jaw in a third position;

FIG. 18 is a perspective view of a portion of the approximation device of FIG. 2;

FIG. 19 is a perspective view of a fastener of an approximation device according to some embodiments;

FIG. 20 shows the fastener of FIG. 19 in a fastener holder according to some embodiments; and

FIG. 21 illustrates a method of using an approximation device according to some embodiments.

### DETAILED DESCRIPTION

An orofacial cleft is a common birth defect that presents as an opening in either or both of the gums and lip of an infant. In addition to causing facial abnormalities, the defect may cause other health issues, such as feeding, hearing, and/or speech problems. Infants suffering from such an orofacial cleft typically undergo several reconstructive surgeries early in life, in an attempt to resolve the defect as their facial structures are developing. For example, the cleft defect may be surgically repaired in three sequential stages: (1) lip repair at approximately 3 months of age, (2) palate repair at approximately 9 months of age, and (3) alveolus repair at approximately 8-10 years of age.

Prior to the first surgery, upper jaw segments may be approximated to reduce the tension in the infant's lip and nose area, near the defect. For example, the clinician may physically manipulate the cleft upper jaw segments over several weeks or months to reposition the cleft jaw segments and decrease the physical distance between the cleft. In some instances, by approximating the cleft jaw segments, tension on the cleft jaw segments may be reduced, which may allow the clinician to more easily manipulate the segments during one of the surgical procedures. In some instances, by approximating the jaw segments, the clinician may improve the infant's lip and reduce the risk of the cleft lip segments separating post-operatively. In some instances, approximating the jaw segments also may reduce the number of surgical procedures needed.

Various techniques may be used to approximate the cleft jaw segments before surgery. For example, the clinician may affix a piece of tape to the infants face to apply pressure through the lip segments to approximate the cleft jaw segments. Fixed and removable oral devices also may be used to approximate the cleft jaw segments. For example, acrylic plates may be affixed to the upper jaw segments for exerting pressure to move and approximate the segments.

The inventor has recognized that existing approximation devices do not allow the clinician to control the way in which the cleft jaw segments are moved towards each other. For example, some know devices move a first jaw segments toward a second jaw segment by pivoting the first jaw segment toward the second jaw segment. As will be appreciated, movement of such devices occur in a similar manner, such as by pivoting a portion of the device attached to the first jaw segment. In some embodiments, the jaw segments are pivoted towards one another irrespective of the position of the first jaw segment relative to the second segment and to the device. This may cause the infant's jaw to collapse, which may require additional surgeries to correct.

The inventor has also recognized that existing devices are not easy to use. For example, existing devices typically require tools to attach the device to the infant. The infant is also required to be under anesthesia during attachment of some know devices.

In view of the above, the inventor has recognized the benefits of an approximation device that allows the clinician to control the way in which the first and second jaws are moved together, such as before a surgical procedure. For example, in some embodiments, the clinician may translate a first jaw segment (e.g., in a direction toward the second jaw segment) before pivoting the first jaw segment towards the second jaw segment. In other embodiments, the clinician may simultaneously translate and pivot the first jaw segment toward the second jaw segment. As will be appreciated, the device also may be used to only pivot the first jaw segment, if desired. In some embodiments, by controlling movement of the jaw segments, the inventor has recognized that the device may maintain an upward force on the jaw segments, which may minimize or even eliminate the possibility of the jaw collapsing.

The inventor has also recognized the benefits of a device that the clinician can easily operate and that does not require the administration of general anesthesia. For example, in some embodiments, the device may be attached without the use of additional tools (e.g., by hand). In such embodiment, the device may include fasteners (e.g., pins) that can be inserted into the infant's jaw segments when the clinician presses the device against the jaw segments (e.g., via one or more fingers).

Additionally, the inventor has recognized the benefits of a device that the infant can better tolerate post-insertion. For example, advantages may be realized if the device may not disrupt the infant's ability to feed following insertion.

Embodiments of the present disclosure include an approximation device (referred to herein as “the device”) arranged to move first and second cleft segments together. According to an aspect of the present disclosure, the clinician can actuate the device so as to control the manner in which the first cleft segment moves toward the second cleft segment. The first and second cleft segments may include first and second jaw segments and/or first and second lip segments.

In some embodiments, the device includes a first jaw attachable to a first cleft segment and a second jaw attachable to a second cleft segment. In some embodiments the first cleft segment is larger than the second cleft segment and is referred to as a greater cleft segment. In such embodiments, the second cleft segment is smaller than the first cleft segment and is referred to as a lesser cleft segment. In these embodiments, the first jaw of the device may be larger than the second jaw of the device. For example, the first, larger jaw may be attachable to the greater cleft segment and the second, smaller jaw may be attachable to the lesser cleft segment.

As will be appreciated, embodiments having similarly sized cleft segments are also contemplated. In such embodiments, the jaws of the device may be configured to be the same size such that each jaw segment is attached to the same sized jaw. In other embodiments, the larger and smaller jaws of the device may be attachable to the similarly sized jaw segments.

In some embodiments, the first and second jaws are attached to a housing. In some embodiments, each of the first and second jaws may be directly attached to the housing. In other embodiments, the second jaw is attachable to the housing via a rocker arm. In some embodiments, as will be described, the first jaw may be fixedly attached to the housing while the second jaw is movably attached to the housing. In some embodiments, the rocker arm may be moveably attached to the housing to drive movement of the second jaw, as will be described. As will be appreciated, both the first and second jaws may be moveably attached to the housing in some embodiments.

In some embodiments, the device is configured to allow the clinician to control movement of at least the second jaw to move the second cleft segment toward the first cleft segment. For example, in some embodiments, actuating the device may move the second jaw between a first position and a second position. In some embodiments, the first position may include a position of the second jaw when the jaw is first attached to the housing and the device is attached to the infant's jaw segment.

In some embodiments, the second jaw may translate from the first position to the second position. For purposes herein, translating the jaw from the first position to the second position may include moving the second jaw linearly from the first position to the second position. For example, the jaw may move from a first position in the infant's mouth to a second position in the infant's mouth, the second position being closer to the opening of the mouth (e.g., in a direction towards the infant's lips). In some embodiments, the second jaw may translate from the first position to the second position along a predetermined travel path. For example, the device may be arranged to translate the second jaw a prescribed distance. In other embodiments, the device may allow the clinician to determine the extent to which (e.g., the distance) the second jaw translates. For example, for some infants, the jaw segment may need to be translated a greater distance than in other infants, where only a small amount of linear translation is needed.

In some embodiments, the device may be actuated to move the second jaw between the second position and a third position. In some embodiments, the second jaw may be rotated from the second position to the third position. For purposes herein, rotating the jaw from the first position to the second position may include moving the second jaw in an arc about a pivot point from the second position to the third position. In some embodiments, the second jaw may rotate from the second position to the third position along a predetermined travel path. For example, the device may be arranged to rotate the second jaw a prescribed distance along the arc. In other embodiments, the device may allow the clinician to determine the extent to which the second jaw rotates. For example, for some infants, the jaw segment may need to be rotated more than in other infants.

In some embodiments, the pivot point may be moveable as the first jaw is translated between the first and second positions. In some embodiments, the pivot point may be positioned closer to a back of the infant's mouth, such as if little to no translation is required. In other embodiments, the pivot point may be positioned closer to a middle or even front of the infant's mouth, such as if a greater amount of linear translation is required.

As will be appreciated, in some embodiments, the device may be actuated such that the second jaw simultaneously translates and rotates from the first position to the second position. For purposes herein, simultaneously translating and rotating the jaw from the first position to the second position may include moving the second jaw in an arc about the pivot point from the first position to the second position while the pivot point moves linearly from the first position to the second position. As with the above, the second jaw may translate and rotate from the first position to the second position along a predetermined path. The clinician also may determine the extent to which the jaw rotates and/or translates.

As will be further appreciated, in still other embodiments, the clinician may combine or even alternate between rotation and translation of the jaw segments. For example, after the jaw segment is moved from the second position to the third position (e.g., via rotation) the clinician may move the jaw segment from the third position to a fourth position. In some embodiments, movement between the third and fourth positions may include translation, translation and rotation, or simply more rotation, as required by the infant.

Although embodiments are shown and described in which a clinician (e.g., a doctor, nurse, surgeon, or other medical professional) activates the device to move the first jaw segment towards the second jaw segment, the device may be used by other individuals in other embodiments. For example, the infant's parent(s) may be instructed on how to use the device. In some embodiments, the device may be activated by the parent(s) at home, under instruction from the clinician, after insertion of the device by the clinician.

In some embodiments the device includes an actuator arranged to move at least the second jaw relative to the housing (and toward the first jaw). In some embodiments, the actuator is attached to the housing. In some embodiments, the actuator is at least partially disposed in the housing. In some embodiments, the actuator includes a first screw (e.g., a hexagonal set screw) and a nut threadable on the screw. In such embodiments, the second jaw may be connected to the nut. In some embodiments, the second jaw is attachable to the nut via the rocker arm (e.g., via a protrusion received in a corresponding channel). In some embodiments, as the clinician turns the screw (e.g., via a complementary tool), the nut moves along the length of the screw, the second jaw moving with the nut. In some embodiments, the nut is disposed within a channel formed in the housing, the channel arranged to maintain a position of the nut relative to the housing.

In some embodiments, a guide channel may be formed in the housing, with a guide pin being formed on the rocker arm. In such embodiments, the channel may include first and second channel portions, with the guide pin moving between the channel portions as the second jaw translates and rotates. For example, in some embodiments, when the pin moves in the first channel portion the second jaw may translate between the first and second positions. In some embodiments, when the pin moves into and along the second channel portion, the jaw may rotate between the second and third positions. In some embodiments, as the clinician rotates the screw, the pin may travel from the first channel portion to the second channel portion.

In some embodiments, the actuator may include first and second screws, each with a threadable nut that travels along the length of the corresponding screw. In some embodiments, the second jaw may be attached to the first and second screws via the rocker arm. For example, the first and second nuts may include first and second protrusions, respectively, with each of the protrusions being received in a corresponding opening in the rocker arm. In some embodiments, the first pin may serve as the pivot point about which the second jaw may rotate. In some embodiments, the second pin may guide movement of the second jaw, with the second opening.

In some embodiments, when the first screw is turned, the first nut moves along the length of the first screw. Similarly, as the second screw is turned the second nut moves along the length of the second screw. In embodiments in which the clinician turns both screws, the rocker arm and second jaw may translate linearly. In embodiments in which the clinician turns only one screw, the second jaw may rotate about the pivot point.

In some embodiments, the device further includes fasteners to attach the first and second jaws to tissue, such as cleft lip segments and/or jaw segments. In some embodiments, the fasteners are attached without a tool.

In some embodiments, the device includes an audible click to alert the user (e.g., the clinician or parent) that one or both of the screws has been turned. In some embodiments, the screw may provide haptic feedback to the user (e.g., the clinician or parent), when turned a predetermined distance. For example, a clinician may instruct a parent to turn the lock “one click per day” after the infant has been sent home. In some embodiments, the device may include one or more locks to provide resistance against backwards resistance of the screw (e.g., anterior sliding or backwards rotation of the screw). For example, in some embodiments, the click may provide mechanical resistance against backward rotation of the screw. In some embodiments, there may be a natural tendency for the jaw segments to relapse to the original position (e.g., before linear translation or rotation), which may drive anterior sliding or backward rotation of the screw.

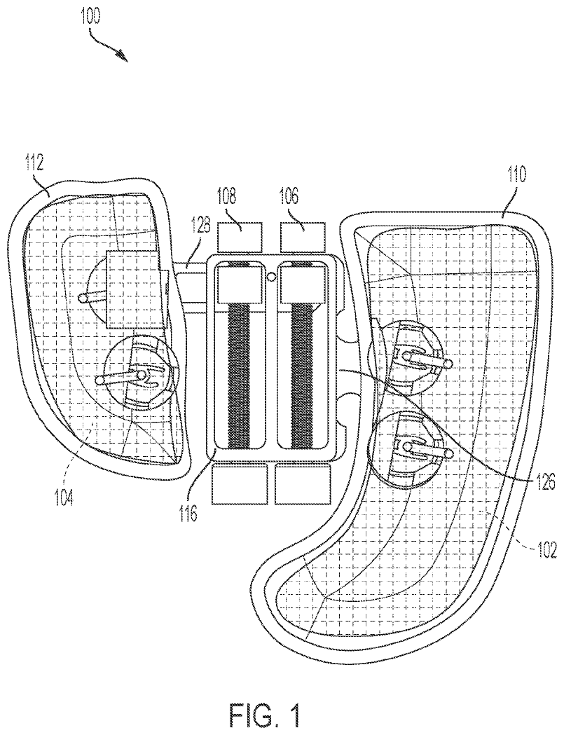

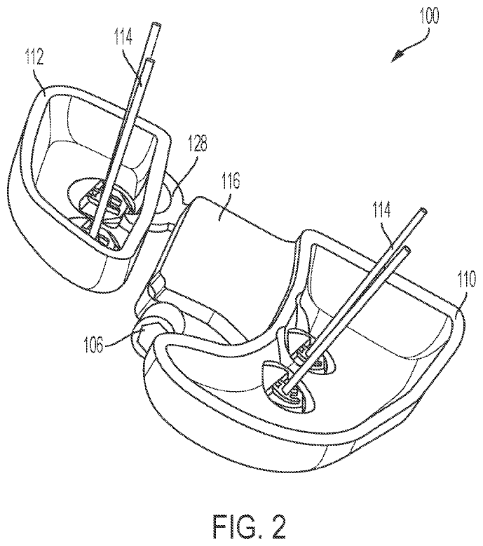

Turning now to the figures, FIGS. 1 and 2 show embodiments of an approximation device 100 arranged to be attached to first and second jaw segments 102, 104 (see FIG. 1) to move the second jaw segment 104 toward the first jaw segment 102. As will be appreciated, movement of the first and second jaw segments may cause movement of first and second tissue portions (e.g., first and second cleft lip segments) towards one another. In some embodiments, as shown in FIG. 1, the device 100 may be attached to the first jaw segment 102 and second jaw segment 104 via a first jaw 110 and a second jaw 112, respectively. In some embodiments, the first jaw segment 102 is larger than second jaw segment 104. As will be appreciated, the second jaw segment 104 also may be larger than and/or of equal size to first jaw segment 102.

In some embodiments, the first jaw 110 and second jaw 112 may be configured to fit the first and second jaw segments 102, 104. For example, the clinician may take an impression of each of the first and second jaw segments 102, 104, and then use the impression to mold the first jaw 110 to fit first jaw segment 102 and the second jaw 112 to fit the second jaw segment 104. As will be appreciated, other manufacturing methods may be used to form the jaws in other embodiments. For example, the clinician may manufacture the first second jaws 110, 112 to fit the first and second jaw segments 102, 104, respectively, via casting, milling, or any other suitable manufacturing method.

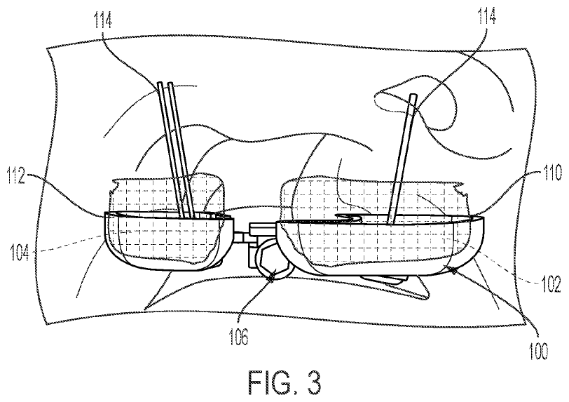

In some embodiments, the device includes fasteners to attach the first and second jaws 110, 112 to the patient (e.g., an infant). For example, the first jaw and second jaws 110, 112 may each include one or more fasteners 114 arranged to secure the jaw to the respective jaw segment. FIG. 3 illustrates the device being attachable to the infant. In some embodiments, the fasteners may include tacks that are inserted into the respective jaw segment. In some embodiments, as shown in FIG. 2, each jaw may include two fasteners for attaching the jaw to the respective jaw segment. Each jaw may have only one fastener or may have more than two fasteners in other embodiments. As will be appreciated, the number of fasteners need not be the same for each jaw. For example, the first (e.g., larger) jaw may have more fasteners than the second (e.g., smaller) jaw in some embodiments. The fasteners may be the same shape and size in some embodiments, although the shape and size may differ from fastener to fastener in some embodiments. Although the fasteners are shown as being pins in these figures, it will be appreciated that the fasteners may have other suitable arrangements in other embodiments.

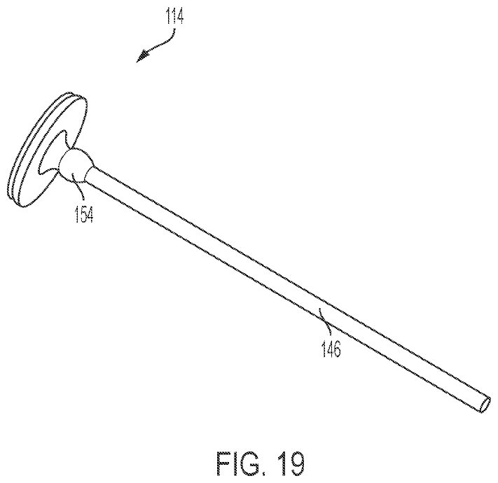

In some embodiments, the fasteners may be permanently attachable to each jaw. The fasteners also may be removably attachable to each jaw. For example, after the jaws have been formed (e.g., by taking impressions of the jaw segments), the fasteners are attachable to the jaw for insertion into the infant. FIG. 19 shows an example of a fastener removed from the jaws. As shown in this view, the fastener may include a pin or tac with a head. As will be appreciated, the head is configured to receive finger pressure to insert the distal end of the pin into the infant.

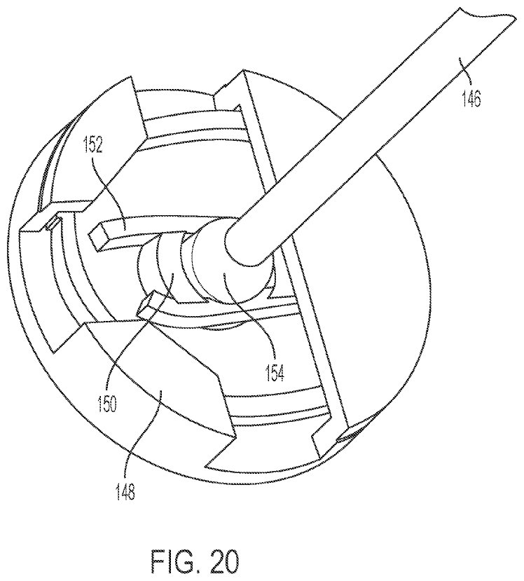

As shown in FIG. 20, the fastener 114 may be received in a fastener holder 148, which may be integrated into the first and/or second jaws 110, 112. In some examples, the pin 146 of the fastener may be inserted into a corresponding opening 150 in the fastener holder 148. In some embodiments, the holder includes a spring wire 152 that, when the clinician inserts the pin 146 into fastener holder 148, is arranged to hold the fastener in the fastener holder. For example, as shown in FIGS. 20, a proximal end of the pin (e.g., near or at the head of the pin) may include a locking region 154 arranged to contact and splay the spring wire outwardly when the clinician is inserting the pin into the holder. As shown in FIG. 20, the diameter of at least a portion of the locking region is larger than a diameter of the pin in some embodiments. The locking region may be tapered or bulbous, although it may have other suitable arrangements. In some embodiments, once the fastener is fully seated in the holder 148 and the locking portion 154 has moved passed the spring wire 152, the spring wire may snap back into place, holding the fastener in the holder. As will be appreciated, the fastener may be held in the holder via other suitable arrangements. For example, the holder may hold the fastener via a press fit, friction fit, or other suitable configuration.

As shown in at least FIG. 1, the first and second jaws 110, 112 are attachable to a housing 116. In some embodiments, the first and second jaws are removably attachable to the housing. For example, after the jaws are made (e.g., via impression of the infant), the jaws are attachable to the housing. As will be appreciated, the jaws may be removed from the housing after the jaw segments have been approximated, and thereafter disposed. In such embodiments, the housing may be configured to be reusable, such that another set of jaws are attachable to the housing. As will be appreciated, the entire device may be disposed of after an approximation.

In some embodiments, the first and second jaws may be directly attached to the housing. In other embodiments, the jaws may be attached to the housing via one or more arms. For example, in some embodiments, as shown in FIGS. 1-3 the second jaw may be attached to the housing via a rocker arm 128. In some embodiments, one or both jaws may be moveable relative to the housing. For example, in some embodiments, the second jaw 112 may be movably attached to housing 116 via the rocker arm 128. In such embodiments, the first jaw 110 may be fixedly attached to the housing. For example, the first jaw may be attached to the housing via a fixed arm 126.

In some embodiments, as shown in FIG. 1, the first jaw 110 is attached to a first lateral side of the housing 116, with the second jaw 112 being attached to a second, opposite lateral side of the housing. In such embodiments, the larger jaw may be attached to the first lateral side of the housing while the smaller jaw is attached to the second lateral side of the housing.

As will be appreciated, during approximation, the smaller jaw segment may be moveable towards the larger jaw segment, however, the smaller jaw segment may be on either side of the infants mouth. In some embodiments, the clinician may orient the device and jaws such that the smaller jaw (e.g., jaw 112) may be moveably attached to the housing no matter the location of the smaller jaw segment. For example, in some embodiments, the housing may be inverted such that the rocker arm is located on the first lateral side, opposite to what is shown in FIG. 1. In such embodiments, the jaws may be attachable to either side of the housing, and be moveable or fixed relative to the housing, as desired. In such embodiments, the second jaw may be attachable to the first lateral side, while the first jaw is attachable to the second lateral side.

According to aspects of the present disclosure, the device is arranged to selectively move first and second jaw segments towards one another. In some embodiments, the device is arranged to move the second jaw segment towards the first jaw segment. For example, in some embodiments, the device 100 may allow the clinician to selectively translate or pivot the second jaw 112 relative to first jaw 110 (and/or to the housing) to translate and/or rotate the second jaw segment relative to the first jaw segment, as needed.

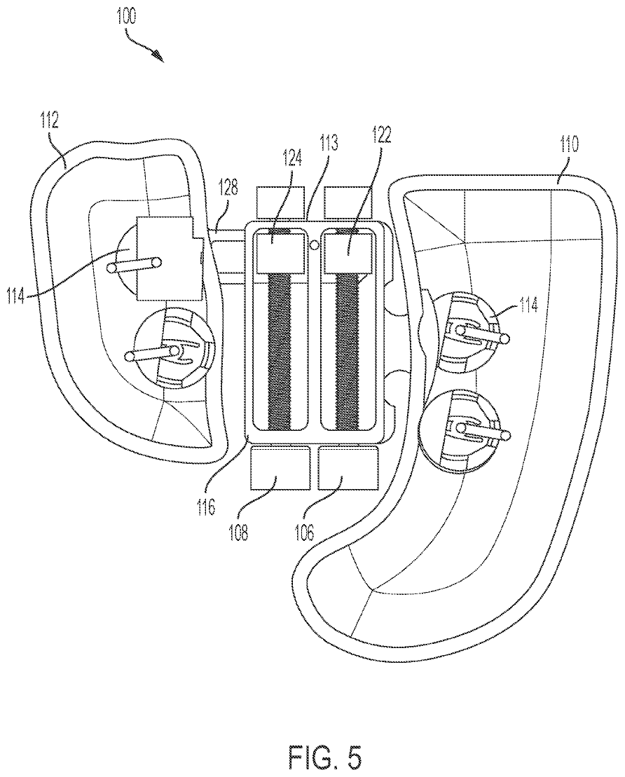

FIGS. 5-7 illustrate an exemplary travel path of the second jaw towards the first jaw during an approximation of first and second jaw segments. In FIG. 5, the second jaw 112 is shown in a first position. In some embodiments, the first position may be a starting position of the second jaw. In some embodiments, the starting position may include the position of the second jaw when the second jaws are initially attached to the housing and the device is attached to the infant. As shown in FIG. 5, in some embodiments, when the second jaw 112 is in the starting position, the rocker arm 128 may be located at or near a first end 113 of the housing. As will be appreciated, upon insertion of the device into the infant's mouth, the first end of the housing may be positioned near a middle or back of the infant's mouth. For example, the first end of the housing may be located further away from the opening of the infant's mouth as compared to the second, opposite end 115 of the housing. In some embodiments, in the starting position, the rocker arm extends substantially perpendicular to a longitudinal axis of the housing. In some embodiments, the rocker arm extends substantially perpendicular to a longitudinal axis of one or more screws 108, 106 in the housing.

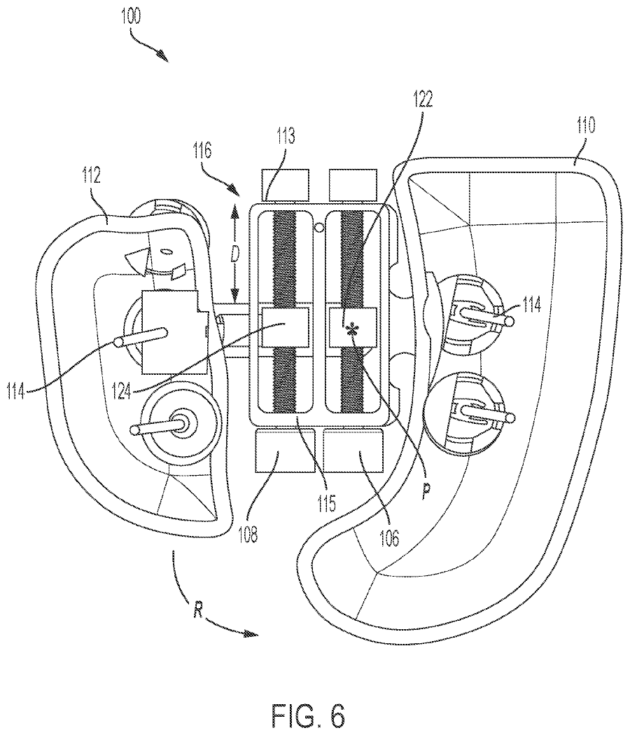

FIG. 6 shows the device with the second jaw 112 in a second position. In some embodiments, as shown in this view, the second jaw is translated from the first position (see FIG. 5) to this second position. For example, the jaw may be moved in a linear direction away from the first end 113 of the housing. In an illustrative example, the jaw has been moved a distance D from the first end 113 of the housing. As will be appreciated, movement of the jaw away from the first end of the housing includes moving the jaw towards a front of the infant's mouth.

In some embodiment, the distance D moved by the second jaw from the first position to the second position may be selected by the clinician. For example, in some embodiments, the clinician may selectively activate the device until the jaw has travelled (and the corresponding cleft jaw segment has moved) the desired amount. In other embodiments, the distance moved by the second jaw from the first position to the second position is predetermined. For example, the jaws may move a prescribed distance from the first end of the housing before being able to move in another direction (e.g., by rotation).

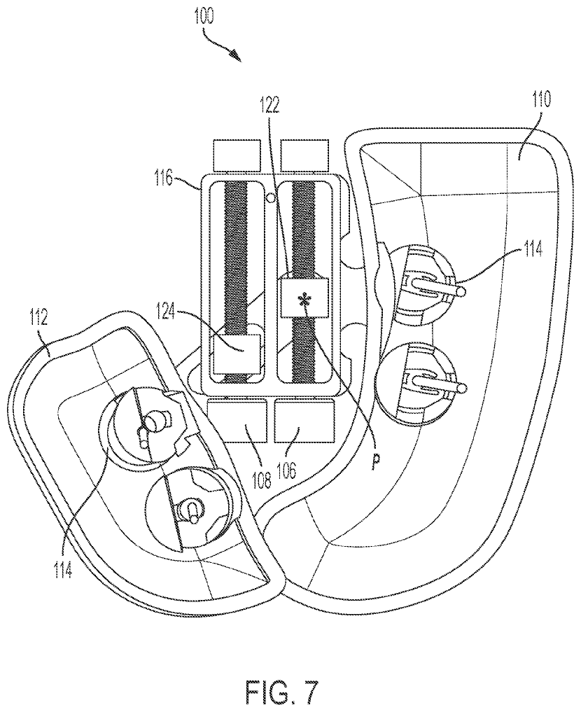

FIG. 7 shows the device with the jaw in a third position. In some embodiments, the jaw is rotated (see the arrow labeled R in FIG. 6) about a pivot point P (see FIG. 6). As shown in FIG. 6, the jaw may rotate in a direction towards the housing and/or towards the first jaw. As with the above, in some embodiments, the device may be arranged such that the clinician may determine the degree to which the second jaw rotates relative to the housing (and the first jaw). In other embodiments, as will be described, the degree of rotation may be preset by the device. For example, in some embodiments, the device rotates the second jaw along a prescribed path.

Although the device is shown as only translating the second jaw relative to the housing (and/or the first jaw) in some steps, and only rotating the second jaw relative to the housing (and/or the first jaw) in some steps, the device may be arranged such that the second jaw may be translated and rotated at the same time. In such embodiments, the second jaw may rotate towards the first jaw as the second jaw translates (and the pivot point moves away from the first end of the housing).

As will be appreciated, although only the second jaw is shown as being moveable relative to the housing (and the first jaw), in other embodiments only the first jaw may be moveable relative to the housing (and the second jaw). In still other embodiments, both the first and second jaws may be moveable relative to the housing and to each other.

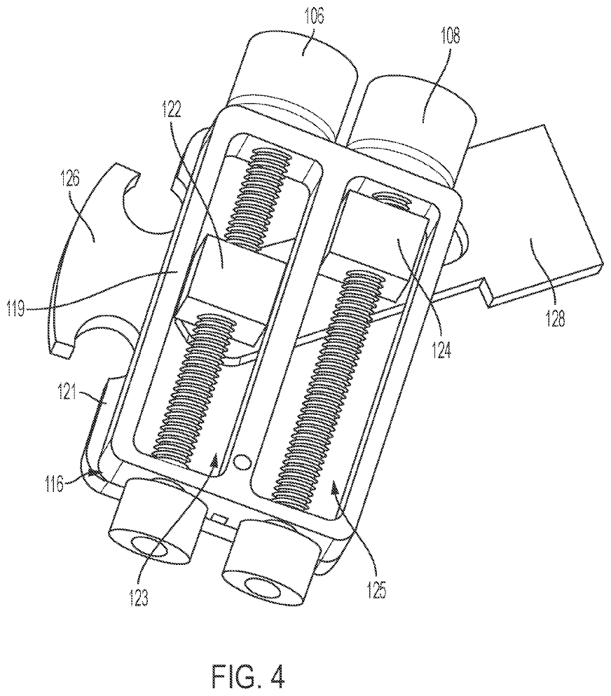

In some embodiments, the device includes an actuator arranged to move at least the second jaw 112 relative to housing 116 (and to the first jaw 110). In some embodiments, the actuator is attached to the housing. In some embodiments, the actuator may be at least partially disposed in the housing. In some embodiments, the housing includes a body 119 and a cover 121. In some embodiments, the cover is fixedly attached to the body (see, e.g., FIG. 4). In some embodiments the cover includes a cover plate.

In some embodiment, as shown in at least FIGS. 1 and 4-7, the actuator includes first and second screws 106, 108 and corresponding first and second nuts 122, 124, respectively, arranged to drive movement of the second jaw (e.g., via the rocker arm). For example, the actuator may include first and second hexagonal set screws that can be actuated by the clinician to drive movement of the jaws. As shown in FIG. 4, each nut may be threaded on the respective screw, and arranged to move along a length of the screw as the clinician turns the screw.

In some embodiments, each of the first and second screw 106, 108 are disposed within the housing. For example, as shown in FIG. 4, each screw may extend in a respective channel 123, 125 formed in the body of the housing. In some embodiments, each channel is substantially rectangular in shape. As shown in this view, each of the screws may extend through first and second openings in the housing, the openings being adjacent to the channels. In some embodiments, each of the first opening is formed in a wall at the first end of the housing and each of the second opening is located in a wall at the second end of the housing.

As shown in FIG. 4, each nut also may be disposed in the channel 123, 125 of the housing. In some embodiments, a width of the channel corresponds to a width of the nut. In some embodiments, the channels are arranged to minimize or event prevent lateral movement of the jaws. For example, in some embodiments, the nut may move linearly within the channel (e.g., between first and second positions), but not move, or may only minimally move, in a lateral direction. The channels also may be arranged to prevent the nuts from rotating when the clinician turns one or both of the screws.

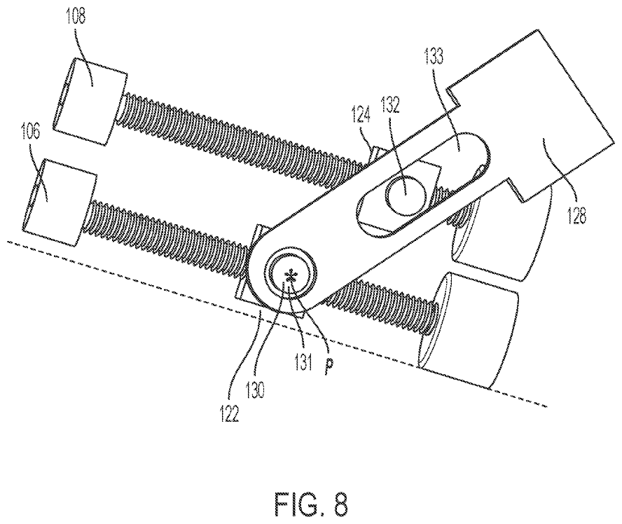

As shown in FIG. 8, the rocker arm may be attachable to the first and second screws, and may cooperate with the first and second screws to drive movement of the second jaw. For example, in some embodiments, the first and second nuts 122, 124 are each configured to interface with rocker arm 128. In some embodiments, the first nut 122 includes a first protrusion 130 configured to fit in a first opening 131 in rocker arm 128. In some embodiments, a shape of the first opening 131 corresponds to a shape of the protrusion 130 on first nut 122. For example, the first protrusion and opening may each be circular in shape. As will be appreciated, the protrusion and opening may have other suitable shapes, such as square, oval, triangular, other polygonal or other shape. In some embodiments, the first protrusion 130 forms the pivot point about which rocker arm 128 (and second jaw) may rotate.

In some embodiments, the second nut 124 has a second protrusion 132 configured to fit in a second opening 133 in rocker arm 128. In some embodiments, the second opening 133 includes an elongate opening in which the second protrusion is moveable while the second jaw pivots. For example, the second protrusion may move within the second opening when the rocker arm pivots about the pivot point P to rotate the second jaw. In some embodiments, second channel may act as a guide channel for guiding movement of second jaw. For example, in some embodiments, the second opening is arranged to control the degree to which the second jaw rotates. For example, when the second protrusion hits one of the ends of the second opening, the second jaw is not able to further rotate.

In some embodiments, the rocker arm is sandwiched between the cover and the first and second nuts, with the first and second protrusions being received in the first and second openings of the rocker arm.

As shown in FIG. 5, when the second jaw is in the first position, the first and second nuts are positioned at the first end 113 of the housing 116. As will be appreciated, the first and second nuts may be located near the first end or even a distance from the first end in the first position in other embodiments. To move the second jaw 112 relative to house and first jaw 110, the clinician may turn first the first and/or second screw 106, 108, causing the first and/or second nuts to move in a direction away from the first end of the housing. In some embodiments, the first nut 122 is configured to travel a length proportional to a distance that first screw 106 is turned. Similarly, the second nut 124 may be configured to travel a length proportional to a distance that second hexagonal set screw 108 is turned.

In some embodiments, when the first and second screws 108 are turned in the same direction at the same rate, the first and second nuts each travel the same distance along the length of each of the first and second screws. The rocker arm 128 and second jaw move along with the first and second nuts, in a direction away from the first end of the housing. FIG. 6 shows the device 100 with second jaw 112 in the second position, after the second jaw has been linearly translated away from the first end of the housing and towards the first jaw 110 (a distance D).

In embodiments in which the clinician turns the first or second screw 106, 108 individually, only the first or second nut will move along the length of the corresponding screw, (e.g., in a direction away from the first end of the housing). In such embodiments, the second protrusion on the second nut will move in the second opening in the rocker arm, and the rocker arm may pivot about the pivot axis P. This, in turn, may cause the second jaw to pivot towards the first jaw.

As will be appreciated, a clinician may pivot and translate the second jaw at the same time in some embodiments. For example, in some embodiments, the clinician may turn the first and second screws at the same time, but at different rates. In such embodiments, the second jaw may pivot about the pivot axis while the pivot axis translates linearly away from the first end of the housing.

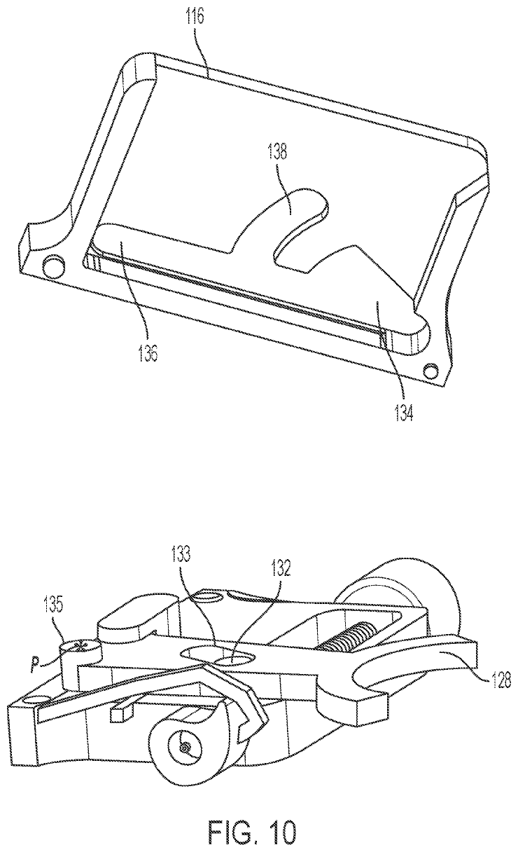

FIGS. 2 and 9-13 illustrate another arrangement of the actuator of the approximation device. As shown in these views, the actuator may again have screw and nut arranged to drive movement of the second jaw. For example, as with the above, the nut may be threadable on the screw, and arranged to move along the length of the screw as the clinician turns the screw. As with the above, the nut may include a protrusion 132 that is received in an elongate channel 133 in the rocker arm (see FIG. 10). As the rocker arm pivots, the protrusion may move in the channel.

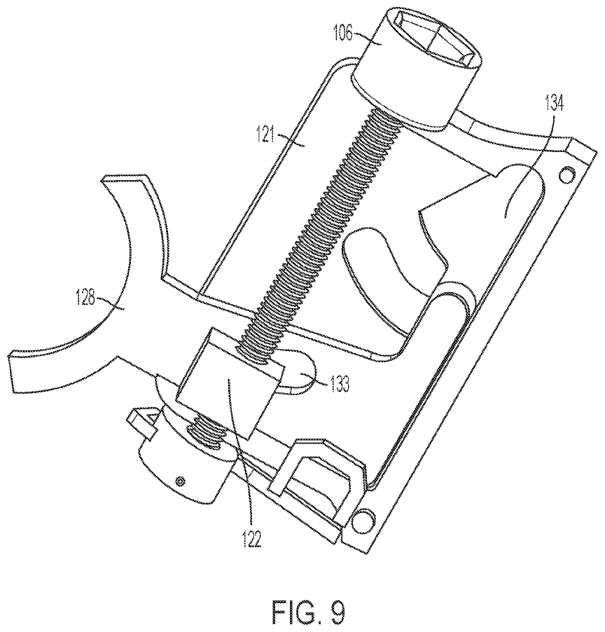

In some embodiments, as shown in FIGS. 9 and 10, the rocker arm may include a guide pin 135 that is received in a guide channel 134 formed in the cover 121 of the housing. In some embodiments, the guide channel is arranged to guide movement of the second jaw. For example, in some embodiments, the guide channel is arranged to guide the second jaw along a prescribed path. In some embodiments, as shown in FIG. 10, the channel may include a first channel portion 136 and a second channel portion 138. In some embodiment, as shown in FIG. 10, the first channel portion is substantially parallel to a longitudinal axis of the cover plate. In some embodiments, the second channel portion is angled relative to the first channel portion. The second channel portion may be curved.

In some embodiments, when the clinician turns the first screw 106, the guide pin of the rocker arm 128 travels along the first channel portion 136 such that the nut, rocker arm, and the second jaw translate linearly in a direction away from the first end of the housing. In such embodiments, once the pin has travelled the prescribed distance the guide pin may enter the second channel portion and the rocker arm may rotate as the clinician turns the first screw 106.

In some embodiments, the width of the channel also corresponds to the width of the guide pin in order to minimize or even prevent undesired lateral movement of the jaw relative to the housing as the jaw translates.

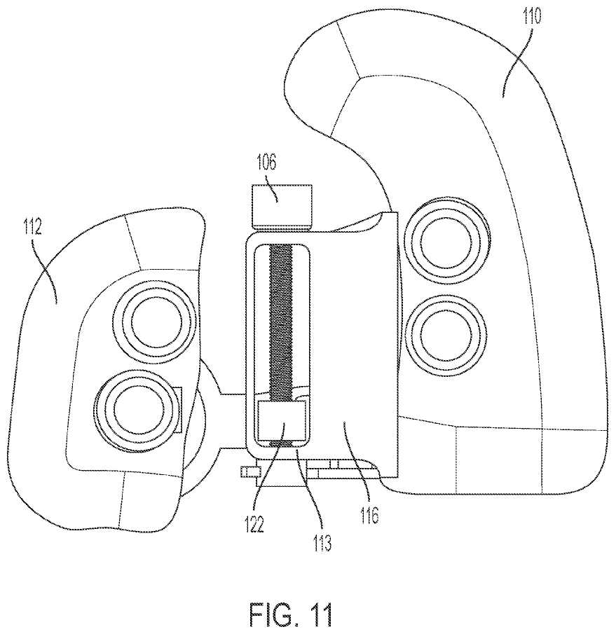

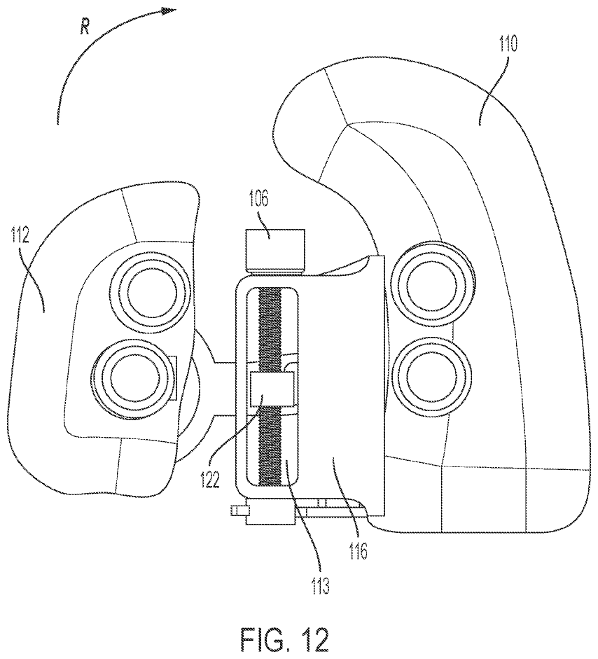

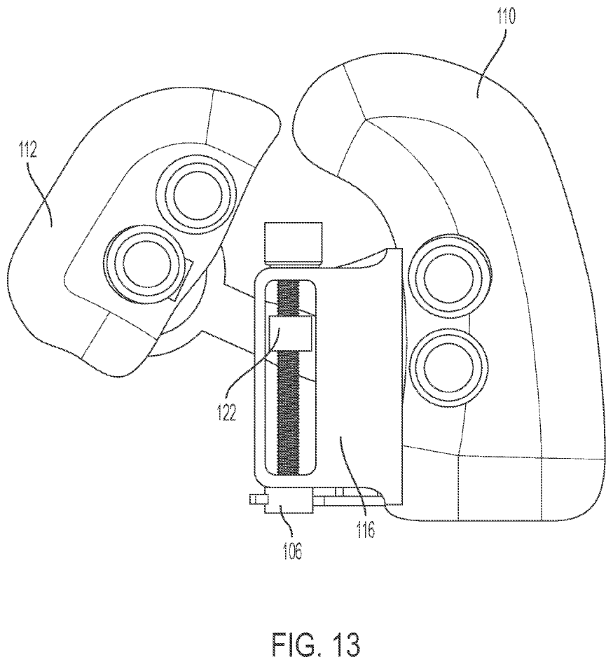

FIGS. 11-13, illustrate movement of the second jaw 112 as the clinician rotates the screw (e.g., between the first and second positions, and then between the second and third positions) FIG. 11 shows the device in the first position. FIG. 12 shows the device after the clinician has turned the screw 106 and the second jaw 112 has translated in a linear direction away from the first end 113 of the housing, to the second position. FIG. 13 shows the device after the second jaw 112 has rotated from the second position to the third position, about the pivot point P (see FIG. 10).

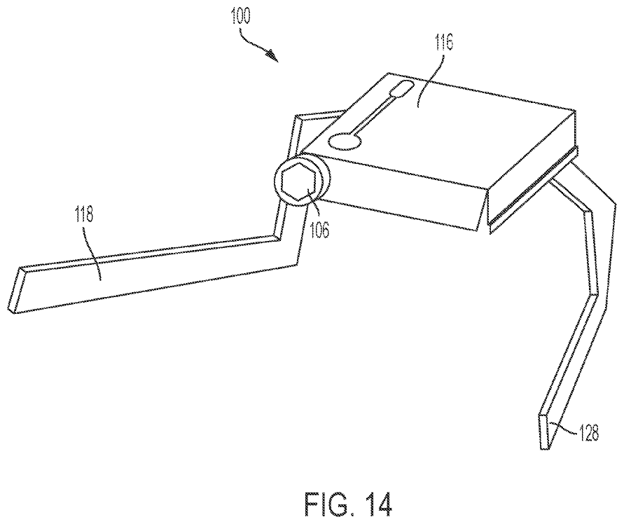

FIG. 14-17 illustrate another arrangement of the actuator of the approximation device. As with the above, and as shown in these views, the approximation device may include a screw that the clinician may turn to drive motion of the second jaw (e.g., via rocker arm 128). In some embodiments, a driver arm 140 is threadable on the screw and is arranged to travel along a length of the screw as the screw is turned. In some embodiments, movement of the driver arm pushes the rocker arm, which causes the second jaw 112 to move relative to the housing.

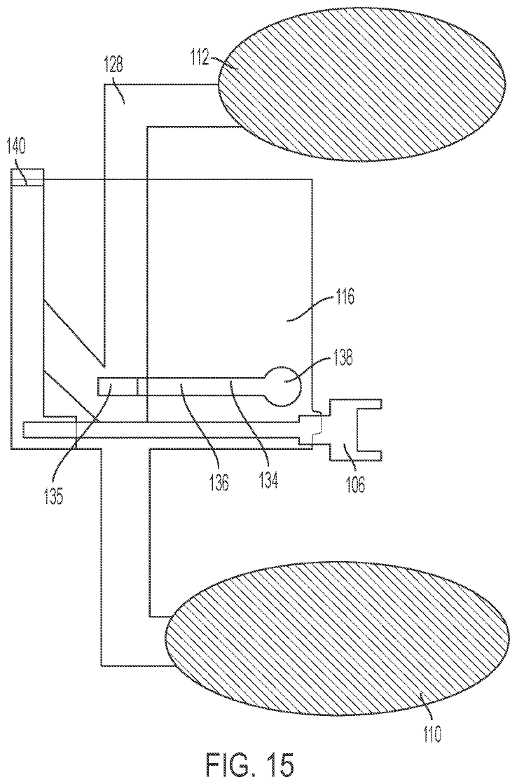

Similar to the above, a guide channel 134 may be formed in the housing to guide movement of the second jaw. As shown in FIG. 15, the guide channel may be formed in the body of the housing, although the guide channel also may be formed in another suitable portion of the housing (e.g., in the cover). In some embodiments, the guide channel has first and second 136, 138 channel portions. In some embodiments, the second channel portion is located at a distal end of the first channel portion. In some embodiments, a width of the channel corresponds to a width of a guide pin 135 on the rocker arm such that lateral movement of the rocker arm (and the jaw) may be minimized and/or prevented. In some embodiments, the second channel portion is wider than the first channel portions such that the guide pin may rotate freely in the second channel portion.

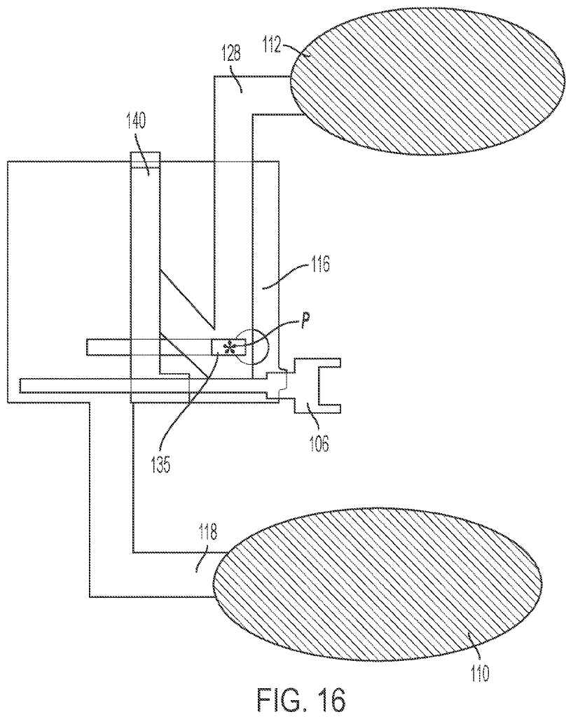

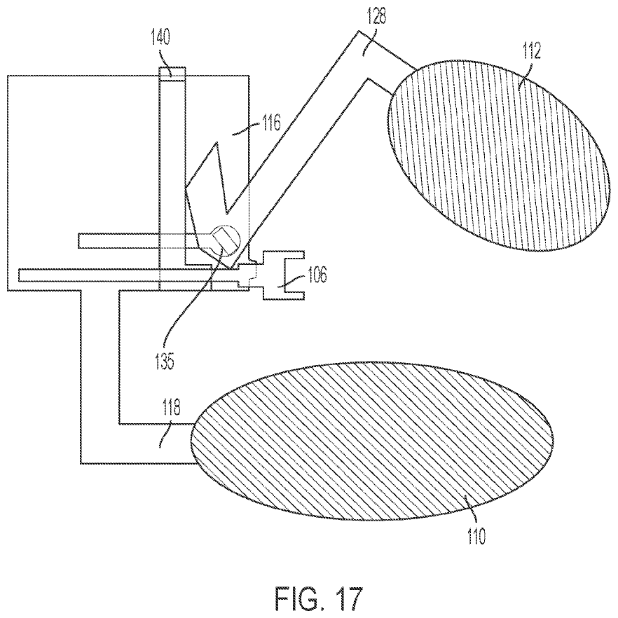

As will be appreciated in view of the above, when the clinician turns the screw and the guide pin is located in the first channel portion, the rocker arm 128 may translate in a direction away from the first end 112 of the housing. FIG. 16 shows the rocker arm after linear translation. Once the guide pin reaches the second channel portion, the guide pin may rotate, causing the rocker arm (and second jaw) to pivot about the pivot axis. FIG. 17 shows the third position, after pivoting of the rocker arm (and second jaw). Similar to the above, the guide pin may define the pivot axis in some embodiments (see FIG. 16). In some embodiments, the drive arm also may stop backward movement of the second arm after rotation has been completed.

Although embodiments are shown and described as using hexagonal set screws, the screws may have other suitable arrangements in other embodiments. For example, the screws 106 and 108 may be Philips screws, slotted screws, square screws, torx screws, or any other suitable configuration. As will be appreciated, other suitable actuators for translating and/or rotating the second jaw may be used in other embodiments.

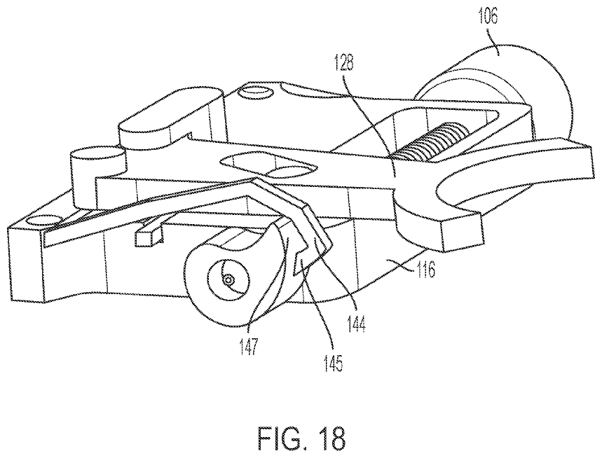

In some embodiments, as shown in FIG. 18, the device 100 may include a lock 144, which may be configured to prevent backward movement of a jaw (e.g., to the second, first, or other position). In some embodiments, the lock 144 may prevent the screw 106 from turning in a direction opposite to that used to move the nut in a direction away from first side of the housing.

In some embodiments, the lock is spring biased into a locked position (see FIG. 18). In some embodiments, the spring may be a leaf spring, a coil spring, a bow spring or any other suitable configuration. In some embodiments, the lock is arranged to contact a knob (e.g., via a contact surface 145) attached to the screw. In some embodiments, when the clinician turns the screw, the knob may splay the lock outwardly. Once an engagement surface 147 of the knob has moved past the lock, the lock my snap back into the locked position. In some embodiments, the lock is arranged to make an audible click, alerting the user (e.g., the clinician or parent), that the screw has been turned a full rotation.

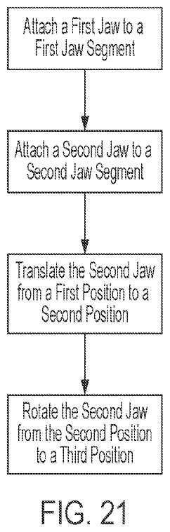

In some embodiments, the disclosure may be embodied as a method of approximating jaw segments and/or tissue portions, the steps of which are outlined in FIG. 21. In some embodiments, the method includes attaching the first jaw 110 to jaw segment. Then, the clinician may attach the second jaw 112 to the second jaw segment. The clinician may then actuate the device to translate second jaw 112 from a first position to a second position. The clinician may then actuate the device to rotate the second jaw 112 from the second position to a third position. As will be appreciated, the translation and/or rotation steps may be performed with one of the above-described actuators. In some embodiments, some of the steps may be performed simultaneously. For example, in some embodiments, the second jaw may be rotated and translated at the same time. In some embodiments, after the method of approximating, a surgical procedure may be performed, whereby the tissue portions (e.g., cleft lip portions) associated with the first and second jaw segments may be attached to each other.

Although the device is shown and described for use with infants to repair a cleft defect, the device may be used to approximate tissue and/or jaw segments in children and/or adults.

As will be further appreciated, although the device is described as being used prior to a surgical procedure, it will be appreciated that the device may be used during a surgical procedure in some embodiments.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. An approximation apparatus comprising:
a first jaw attachable to a first jaw segment;
a second jaw attachable to a second jaw segment, the second jaw segment being smaller than the first jaw segment; and
a housing;
wherein the second jaw is movably attached to the housing, the second jaw configured to move the second jaw segment toward the first jaw segment;
wherein the second jaw is configured to translate between a first position and a second position during a first segment of travel of the second jaw;
wherein the second jaw is configured to at least rotate between the second position and a third position during a second segment of travel.

2. The apparatus of claim 1, wherein the first jaw is fixedly attached to the housing

3. The apparatus of claim 1, wherein the second jaw is attached to the housing via a rocker arm, wherein the rocker arm is moveably attached to the housing.

4. The apparatus of claim 3, wherein the second jaw is fixedly attached to the rocker arm.

5. The apparatus of claim 3, further comprising an actuator arranged to move the second jaw from the first position to the second position and from the second position to the third position.

6. The apparatus of claim 1, wherein the actuator includes:
a first screw; and
a first nut threadable on the first screw, the first nut arranged to travel along a length of the first screw;
wherein the first nut is connected to the rocker arm.

7. The apparatus of claim 1, wherein the first nut includes a first protrusion received in a first opening of the rocker arm.

8. The apparatus of claim 7, wherein the actuator further includes:
a second screw; and
a second nut threadable on the second screw, the second nut arranged to travel along a length of the second screw;
wherein the second nut is connected to the rocker arm.

9. The apparatus of claim 8, wherein the second nut includes a second protrusion received in a second opening in the rocker arm.

10. The apparatus of claim 5, wherein the actuator is at least partially disposed in the housing.

11. The apparatus of claim 6, wherein the rocker arm includes a guide pin, the guide pin received in a channel formed in one of a cover and a body of the housing

12. The apparatus of claim 1, wherein each of the first and jaws include one or more fasteners arranged to attach the jaw to the respective jaw segment.

13. The apparatus of claim 12, wherein the one or more fasteners are removably attachable to the respective jaw.

14. An approximation device comprising:
a first jaw attachable to a first jaw segment;
a second jaw attachable to a second jaw segment, the second jaw arranged to move the second jaw segment toward the first jaw segment; and
a housing, wherein the first jaw is fixedly attached to the housing and the second jaw is attached to the housing via a rocker arm, the rocker arm being moveable relative to the housing;
wherein the rocker arm is configured to translate the second jaw from a first position to a second position during a first segment of travel of the second jaw, and to at least rotate the second jaw from the second position to a third position during a second segment of travel.

15. The device of claim 14, wherein the second jaw is fixedly attached to the rocker arm.

16. The device of claim 14, further comprising an actuator arranged to move the second jaw from the first position to the second position, and from the second position to the third position.

17. The device of claim 16, wherein the actuator includes a first screw and a first nut threadable on the first screw and arranged to travel along a length of the first screw, the rocker arm being connected to the first nut.

18. The device of claim 17, wherein the actuator includes a second screw and a second nut threadable on the second screw and arranged to travel along a length of the second screw, the rocker arm being connected to the second nut.

19. The device of claim 17, wherein the rocker arm includes a guide pin received in a guide channel in the housing.

20. The device of claim 14, wherein the rocker arm is at least partially disposed in the housing.

21. A method of approximating first and second and jaw segments via an approximation device having first and second jaws attached to a housing, the method comprising:
attaching a first jaw to a first jaw segment, the first jaw being attached to a housing;
attaching a second jaw to a second jaw segment, the second jaw being moveably attached to the housing;
translating the second jaw relative to the housing to move the second jaw from a first position to a second position; and
pivoting the second jaw relative to the housing to move the second jaw from the second position to a third position.

22. The method of claim 21, wherein the step of pivoting is performed after the step of translating.

23. The method of claim 21, further comprising, before the step of attaching the first and second jaws, forming the first and second jaws.

24. The method of claim 21, wherein translating includes moving the second jaw linearly in a direction away from a first side of the housing.

25. The method of claim 21, further comprising, before the step of translating, actuating the device, the step of actuating including:
rotating a first screw;
moving a first nut along a first length of the first screw, the second jaw being attached to the first nut via a rocker arm.

26. The method of claim 25, further comprising, before the step of pivoting, rotating the first screw, and moving the first nut along a second length of the first screw.

27. The method of claim 26, further comprising, pivoting the rocker arm relative to the housing.

28. The method of claim 27, wherein pivoting the rocker arm includes moving a protrusion of the rocker arm in a channel formed in the housing.
